(19) United States
(12) Patent Application Publication
(10) Pub. No.: US 2012/0209406 A1
(43) Pub. Date: Aug. 16, 2012

(54) Foot Plate Device for An Artificial Foot

(75) Inventors: Jian-Yu Chen, New Taipei City (TW); Chia-Chung WU, Taipei City (TW); Wun-Ya Gao, New Taipei City (TW); Jian-Hong Lin, New Taipei City (TW)

(73) Assignee: TEH LIN PROSTHETIC & ORTHOPAEDIC INC., New Taipei City (TW)

(21) Appl. No.: 13/396,491

(22) Filed: Feb. 14, 2012

(30) Foreign Application Priority Data
Feb. 16, 2011 (TW) 100202827

(51) Int. Cl. A61F 2/66

(52) U.S. Cl. 623/55

(57) ABSTRACT

A foot plate device is connected to a prosthetic lower leg of an artificial foot, and includes a flexible foot plate. The foot plate has a front plate section, an inclined intermediate plate section extending rearwardly and upwardly from a rear end of the front plate section, and a rear plate section extending rearwardly from a rear end of the intermediate plate section and generally parallel to the front plate section. The front plate section has at least one open-ended slot formed vertically therethrough and extending across the front plate section along a longitudinal direction of the foot plate, so as to divide the front plate section into two force-receiving plate portions. The rear plate section is connected with the prosthetic lower leg.

### CROSS-REFERENCE TO RELATED APPLICATION

This application claims priority of Taiwanese Application No. 100202827, filed on Feb. 16, 2011.

### BACKGROUND OF THE INVENTION

1. Field of the Invention

This invention relates to an artificial foot, and more particularly to a foot plate device for an artificial foot.

2. Description of the Related Art

Referring to FIG. 1, a conventional foot plate device 1 for an artificial foot is configured as an arched resilient metallic plate, and has a raised front end for supporting and buffering purposes. A problem is encountered by the conventional foot plate device 1. That is, if the left or right side of the resilient metallic plate is moved onto a bumped road surface, the resilience metallic plate will recline to result in unbalance of the artificial foot.

### SUMMARY OF THE INVENTION

The object of this invention is to provide a foot plate device for an artificial foot, which can maintain balance of the artificial foot when it is moved on a bumped road surface.

According to this invention, a foot plate device is connected to a prosthetic lower leg of an artificial foot, and includes a flexible foot plate. The foot plate has a front plate section, an inclined intermediate plate section extending rearwardly and upwardly from a rear end of the front plate section, and a rear plate section extending rearwardly from a rear end of the intermediate plate section and generally parallel to the front plate section. The front plate section has at least one open-ended slot formed vertically therethrough and extending across the front plate section along a longitudinal direction of the foot plate, so as to divide the front plate section into two force-receiving plate portions. The rear plate section is connected with the prosthetic lower leg.

Due to inclusion of the force-receiving plate portions in the foot plate, when only one of the force-receiving plate portions is moved onto a bump on a road surface, the other one of the force-receiving surfaces can still contact the road surface for maintaining balance of the artificial foot, thereby enhancing safety during use of the artificial foot.

### BRIEF DESCRIPTION OF THE DRAWINGS

These and other features and advantages of this invention will become apparent in the following detailed description of the preferred embodiments of this invention, with reference to the accompanying drawings, in which:

FIG. 1 is a perspective view of a conventional foot plate device for an artificial foot;

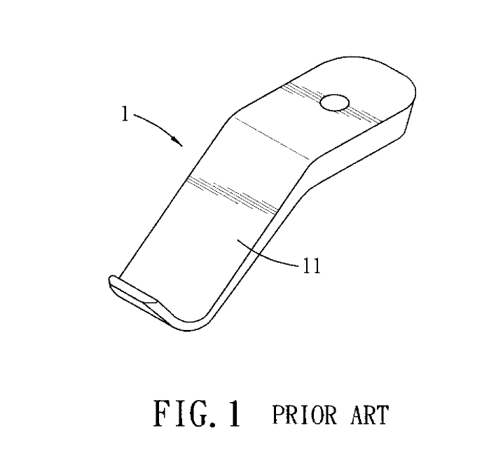

FIG. 2 is an assembled perspective view of the first preferred embodiment of a foot plate device for an artificial foot according to this invention;

FIG. 3 is an assembled perspective view of the second preferred embodiment of a foot plate device for an artificial foot according to this invention;

FIG. 4 is an exploded perspective view of the second preferred embodiment;

FIG. 5 is an assembled perspective view of the third preferred embodiment of a foot plate device for an artificial foot according to this invention; and

FIG. 6 is an exploded perspective view of the third preferred embodiment.

### DETAILED DESCRIPTION OF THE PREFERRED EMBODIMENTS

Before the present invention is described in greater detail in connection with the preferred embodiments, it should be noted that similar elements and structures are designated by like reference numerals throughout the entire disclosure.

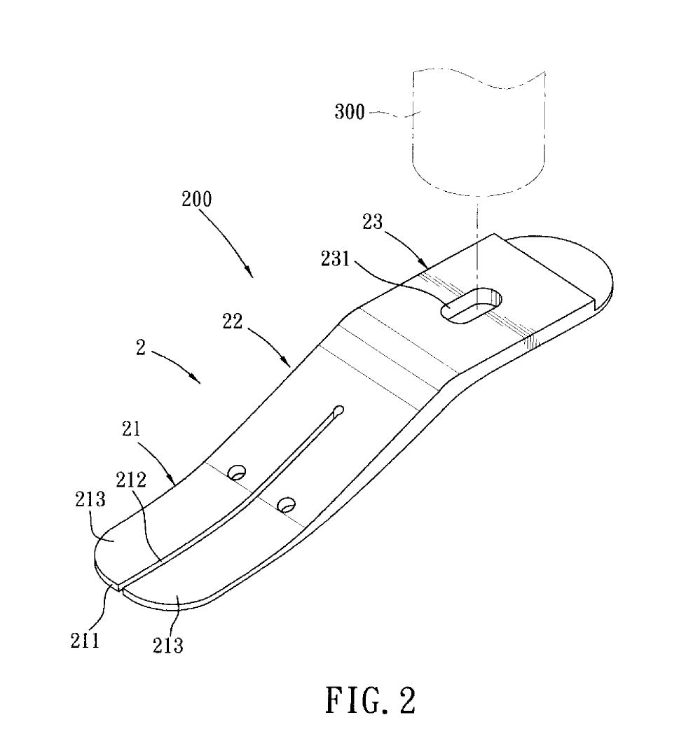

Referring to FIG. 2, the first preferred embodiment of a foot plate device 200 for an artificial foot according to this invention is used to be connected with a prosthetic lower leg 300. The foot plate device 200 is configured as a flexible foot plate 2.

The foot plate 2 has a front plate section 21 substituting for toes, an inclined intermediate plate section 22 extending rearwardly and upwardly from a rear end of the front plate section 21, and a rear plate section 23 extending rearwardly from a rear end of the intermediate plate section 22 and generally parallel to the front plate section 21. The rear plate section 23 has a coupling portion 231 for connection with the prosthetic lower leg 300.

The front plate section 21 has a front end 211, and an open-ended slot 211 that is formed vertically therethrough and that extends rearwardly from the front end 211 and across the front plate section 21 along a longitudinal direction of the foot plate 2, so as to divide the front plate section 21 into two force-receiving plate portions 213 located respectively at two sides of the open-ended slot 212. As such, the flexibility of the force-receiving plate portions 213 is relatively high so that, when only one of the force-receiving plate portions 213 is moved onto a bump on a road surface, the other one of the force-receiving surfaces 213 can still contact the road surface for maintaining balance of the artificial foot, thereby enhancing safety during use of the artificial foot.

Alternatively, the number of the open-ended slot 212 may be increased.

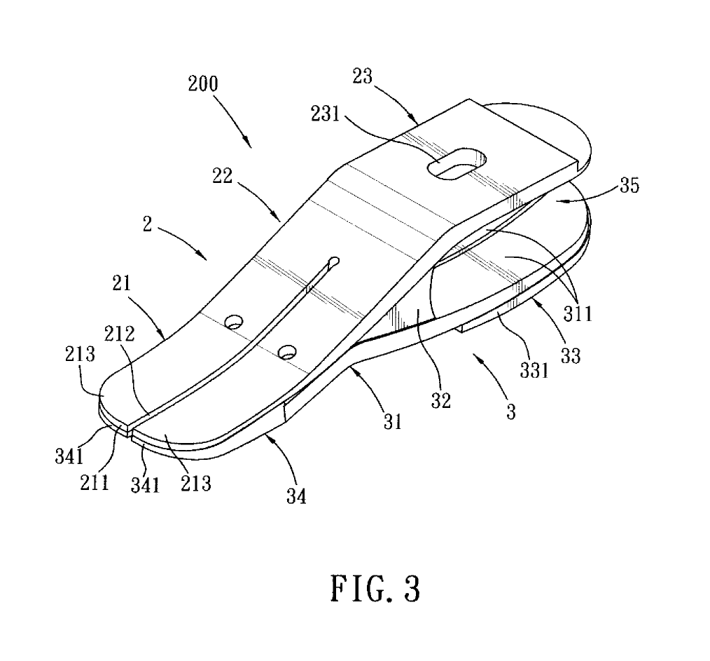

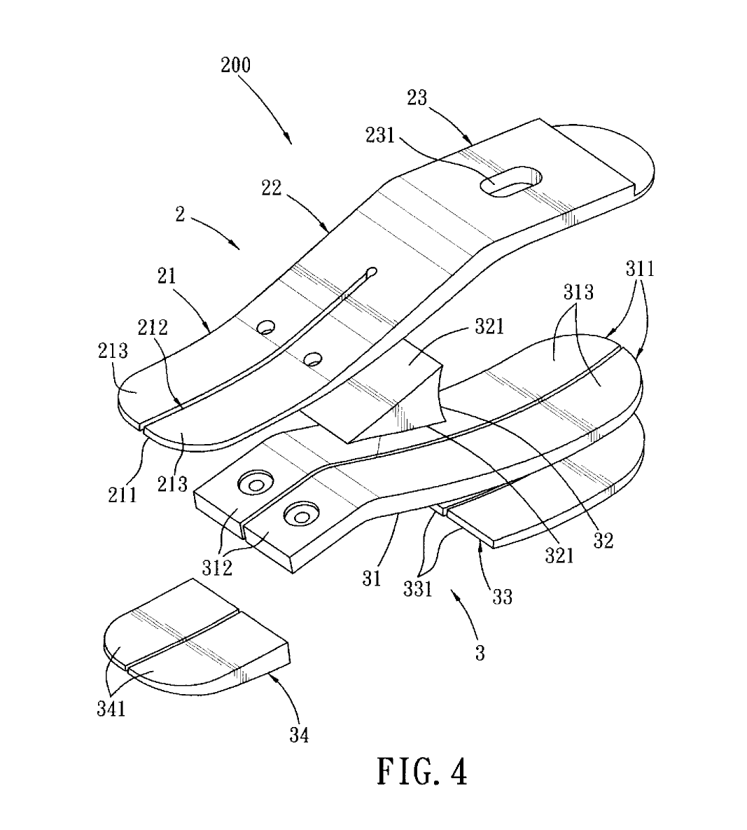

FIGS. 3 and 4 show the second preferred embodiment of a foot plate device 200 for an artificial foot according to this invention, which is similar to the first preferred embodiment except for addition of a supporting plate assembly 3. The supporting plate assembly 3 includes a bottom plate unit 31 secured to and disposed under the foot plate 2, a cushioning member 32 disposed between and in contact with the foot plate 2 and the bottom plate unit 31, a heel plate unit 33 secured to and disposed under a rear end portion of the bottom plate unit 31, and a toe plate unit 34 secured to and disposed under a front end portion of the foot plate 2 and disposed in front of and adjacent to the bottom plate unit 31. As such, the toe plate portion 34 is not connected directly to the bottom plate unit 31, thereby facilitating flexure of the front plate section 21.

The bottom plate unit 31 includes two bottom plates 311 having front ends aligned respectively with the force-receiving plate portions 213. Each of the bottom plates 311 has a front connecting portion 312 disposed at a front end portion thereof and connected fixedly to the foot plate 2, and a curved rear buffering portion 313 extending rearwardly and downwardly from a rear end of the front connecting portion 312. The cushioning member 32 is fixed between the foot plate 2 and a front end of the rear buffering portions of the bottom plates 311. The heel plate unit 33 includes two heel plates 331 connected respectively and fixedly to the curved rear buffering portions 313 of the bottom plates 311. The toe plate unit 34 includes two toe plates 341 connected respectively and fixedly to the force-receiving plate portions 213 of the front plate section 21 and having rear ends abutting respectively against front ends of the front connecting portions 312 of the bottom plates 311.

The cushioning member 32 is configured as a wedge having two interconnected abutting surfaces 321 that abut respectively against the foot plate 2 and the bottom plate unit 31 and that form an acute angle therebetween.

A space 35 is left between the rear plate section 23 of the foot plate 2 and the bottom plate unit 31 such that, when the foot plate device 200 is moved, rear ends of the rear plate section 23 and the bottom plate unit 31 move toward and away from each other, so as to provide a buffering effect.

In this embodiment, the foot plate 2, the bottom plates 311, the heel plates 33, and the toe plates 34 are resilient sheets made of fiber reinforced plastic or carbon fiber, and the cushioning member 32 is made of a plastic material.

In this embodiment, to establish a secure connection, glue is applied between the foot plate 2 and the bottom plates 311, between the foot plate 2 and the cushioning member 32, between the foot plate 2 and the toe plates 34, between the cushioning member 32 and the bottom plates 311, and between the bottom plates 311 and the heel plates 33.

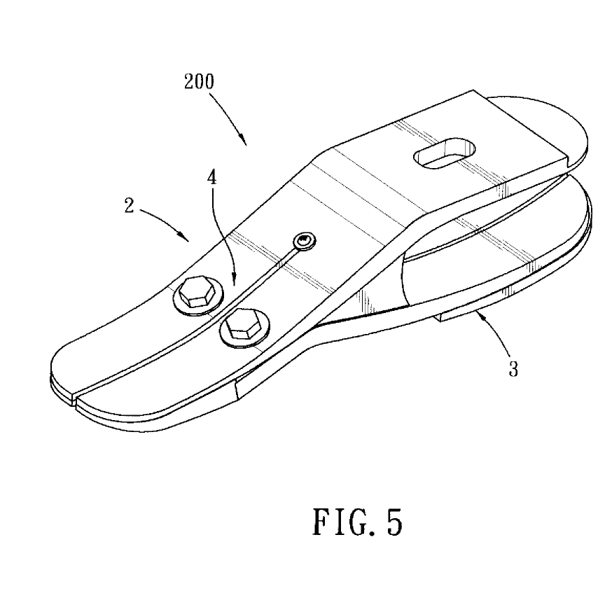

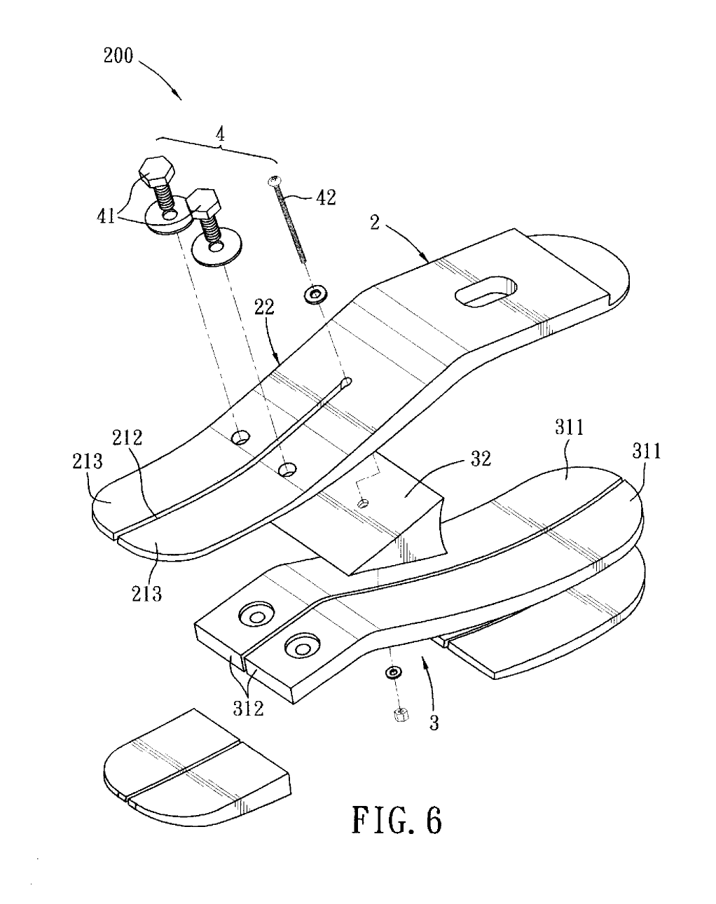

FIGS. 5 and 6 show the third preferred embodiment of a foot plate device 200 for an artificial foot according to this invention, which differs from the second preferred embodiment in the secure connection manner. Unlike the second preferred embodiment, the bottom plates 311 of the supporting plate assembly 3 are connected fixedly to the foot plate 2 by a fastening unit 4. The fastening unit 4 includes two large lock bolt units 41 each extending through the corresponding force-receiving plate portion 213 and the corresponding front connecting portion 312, and a small lock bolt unit 42 disposed behind the large lock bolt units 41 and extending through the open-ended slot 212 and the cushioning member 32 and between the bottom plates 311.

With this invention thus explained, it is apparent that numerous modifications and variations can be made without departing from the scope and spirit of this invention. It is therefore intended that this invention be limited only as indicated by the appended claims.

We claim:

1. A foot plate device adapted to be connected with a prosthetic lower leg, said foot plate device comprising a flexible foot plate, said foot plate having a front plate section, an inclined intermediate plate section extending rearwardly and upwardly from a rear end of said front plate section, and a rear plate section extending rearwardly from a rear end of said intermediate plate section and generally parallel to said front plate section, said front plate section having at least one open-ended slot that is formed vertically therethrough and that extends across said front plate section along a longitudinal direction of said foot plate, so as to divide said front plate section into two force-receiving plate portions, said rear plate section being adapted to be connected with the prosthetic lower leg.

2. The foot plate device as claimed in claim 1, further comprising a supporting plate assembly, said supporting plate assembly including a bottom plate unit secured to and disposed under said foot plate, a cushioning member disposed between and in contact with said foot plate and said bottom plate unit in such a manner that a space is left between said rear plate section of said foot plate and said bottom plate unit, a heel plate unit secured to and disposed under a rear end portion of said bottom plate unit, and a toe plate unit secured to and disposed under said front plate section of said foot plate and disposed in front of and adjacent to said bottom plate unit.

3. The foot plate device as claimed in claim 2, wherein said bottom plate unit includes two bottom plates having front ends aligned respectively with said force-receiving plate portions, each of said bottom plates having a front connecting portion disposed at a front end portion thereof and connected fixedly to said foot plate, and a curved rear buffering portion extending rearwardly and downwardly from a rear end of said front connecting portion, said cushioning member being fixed between said foot plate and front ends of said rear buffering portions of said bottom plates.

4. The foot plate device as claimed in claim 3, wherein said heel plate unit includes two heel plates connected respectively to said curved rear buffering portions of said bottom plates.

5. The foot plate device as claimed in claim 3, wherein said toe plate unit includes two toe plates connected respectively to said force-receiving plate portions and having rear ends abutting respectively against front ends of said front connecting portions of said bottom plates.

6. The foot plate device as claimed in claim 3, wherein said cushioning member is configured as a wedge having two interconnected abutting surfaces that abut respectively against said foot plate and said bottom plate unit and that form an acute angle therebetween.
